(19) United States
(12) Patent Application Publication
(10) Pub. No.: US 2023/0395363 A1
(43) Pub. Date: Dec. 7, 2023

(54) A SIGNAL PROCESSING METHOD AND A MASS SPECTROMETER USING THE SAME

(71) Applicant: KARSA OY, Helsinki (FI)

(72) Inventors: Oskari KAUSIALA, Tampere (FI); Aleksei SHCHERBININ, Helsinki (FI)

(21) Appl. No.: 18/249,668

(22) PCT Filed: Dec. 1, 2020
(86) PCT No.: PCT/FI2020/050808

§ 371 Date: Apr. 19, 2023

(30) Foreign Application Priority Data
Dec. 2, 2019 (FI) 20196044

(51) Int. Cl. H01J 49/00; G16C 20/20; G16C 20/70

(52) U.S. Cl. CPC H01J 49/0036; G16C 20/20; G16C 20/70

(57) ABSTRACT

A signal processing method (100) of a mass spectrometer signal, indicative of mass spectrum of atoms abundant in the mass spectrometer analyzed sample, where the signal processing method comprises: segmentation of the spectra (102), compression of the data points (103), peak finding from the spectra (104), ridge detection (105) at the adjacent peaks represented but the consecutive spectra, peak data processing (106), and identification of peaks (107). The signal processing method is used in a mass spectrometer and a mass spectrometer system.

### TECHNICAL FIELD

This disclosure generally relates to systems and methods for signal processing and, more particularly, to such a signal processing that relates to signal processing of a mass spectrometer to find and identify substances from mass spectra, being represented by the signal being processed.

### BACKGROUND

Advances in the technical field of material sciences, especially in recognition of substances in trace level abundances is a challenging problem. Sometimes small quantities present in the material under an examination may produce distinctive properties to the material as the chemistry, structure or behavior in terms of electro-mechanical, magnetic and/or strength as well as hydraulic or pneumatic properties that may be changed because of the traces, present but barely visible if at all. If a mass spectrometer as such could be used in the material analysis of the abundant species, to track also the trace metal abundances, there might be too much noise as well as something that is not noise as such but would be hiding in the mass spectra in the peaks to hide the small concentration levelled traces under the noise or other, possibly higher peaks of more abundant substances or isotopes thereof.

In general, a high-resolution mass spectrometer can produce hundreds of thousands of data points per spectrum per second. This large data stream poses a signal processing challenge for applications where (near) real-time processing is required.

However, the possibility of coincidence of peaks makes the element composition difficult, and makes the peak associated element identification time consuming and may be not possible at all without a sufficiently fast algorithm to separate and recognize the peaks and the associated elements, to produce information about the compositions of the ions present in the sample.

Embodiments of the disclosure are aimed to solve the difficulties in the conventional mass spectrometer use, but at least to mitigate the consequences caused by the appearing problems.

A signal processing method of mass spectrometer signal, according to one or more embodiments of the instant disclosure, being indicative of mass spectrum of atoms abundant in the mass spectrometer analyzed sample, the signal processing method comprises:
segmentation of the spectra,
compression of the data points,
peak finding from the spectra,
ridge detection, at the adjacent peaks represented by consecutive spectra,
peak data processing, and
identification of peaks.

The signal processing method according to one or more embodiments of the instant disclosure can additionally comprises identifying the chemical information for the substances and their abundances, and/or classification of the findings.

The signal processing method according to one or more embodiments of the instant disclosure comprises supersampling.

The signal processing method according to one or more embodiments of the instant disclosure comprises determining a supersampling factor in accordance of the dictionary matrix sample size.

The signal processing method according to according to one or more embodiments of the instant disclosure comprises determining a Pearson correlation coefficient for the time series of the set of peaks.

The signal processing method according to one or more embodiments of the instant disclosure comprises that wherein the Pearson correlation coefficients between all the peaks are applied as a constraint to match a predefined threshold.

The signal processing method according to one or more embodiments of the instant disclosure, comprises a machine learning algorithm (ML) configured to operate as a feature extraction means in the peak detection and/or target detection.

The signal processing method according to one or more embodiments of the instant disclosure comprises in the method that the measured mass spectrometer spectra are processed by the signal processing method as a solid batch of spectra with a predetermined size to form a solid ensemble of spectra.

The signal processing method according to a according to one or more embodiments of the instant disclosure comprises in the method that the measured mass spectrometer spectra are processed by the signal processing method as a gliding batch of spectra to an ensemble of before measured spectra to add a new spectrum so to increase the predetermined number of the ensemble of spectra. This is suitable in online measurements made by the mass spectrometer.

The signal processing method according to one or more embodiments of the instant disclosure comprises updating at least one of the following: segment number, compression of the segments, the dictionary matrix size, the ridge detection ridge associated data and a peak number.

A mass spectrometer according to one or more embodiments of the instant disclosure is configured to signal processing according to a signal processing method according to one or more embodiments of the instant disclosure.

A non-transitory computer-readable medium according to one or more embodiments of the instant disclosure is configured to store computer-executable instructions which when executed by one or more processors result in performing operations comprising the signal processing method according to one or more embodiments of the instant disclosure.

A mass spectrometer system according to one or more embodiments of the instant disclosure comprises a software in a software block to provide dedicated functionality to the mass spectrometer of the mass spectrometer system, additionally the mass spectrometer system comprising further analyzer hardware to acquire mass spectra, microprocessor for controlling the dedicated functionality of the mass spectrometer with the analyzer hardware, memory and an I/O interface for mediating the control signals of the microprocessor according to the software block control.

The mass spectrometer system according to one or more embodiments of the instant disclosure comprises such a software that comprises computer-executable instructions according to one or more embodiments of the instant disclosure, configured to implement the signal processing method according to one or more embodiments of the instant disclosure.

The mass spectrometer system according to one or more embodiments of the instant disclosure, wherein the mass spectrometer system comprises a wireless information network access point for external control by an external user equipment.

### BRIEF DESCRIPTION OF THE DRAWINGS

FIG. 1 depicts an example of an embodiment algorithm flowchart about a signal processing stages, in accordance with one or more example embodiments of the present disclosure.

FIG. 2 depicts an illustrative diagram for an embodiment as relating to segmentation of an ensemble of mass spectra for peak identification from them, in accordance with one or more example embodiments of the present disclosure.

FIG. 3 depicts an illustrative flow diagram for an arbitrary experimentally defined ion transmission function (peak with a “peak shape”), used as a dictionary atom, in accordance with one or more example embodiments of the present disclosure.

FIG. 4 depicts an illustrative schematic diagram for an illustration of the peak dictionary for one segment of a mass spectrum in an ensemble of mass spectra, showing the atom corresponding to every 10th sample for clarity; each peak is stored as a row into a dictionary matrix, in accordance with one or more example embodiments of the present disclosure.

FIG. 5 depicts illustrative sequences of an ensemble of ten segments of data with the compression algorithm results depicted by the vertical bars at the peaks in the spectra, in accordance with one or more example embodiments of the present disclosure.

FIG. 6 depicts a flow diagram of an illustrative flattened view (FIG. 5) of the results, detected peaks depicted by the vertical lines and cross markers. Four peaks have been detected. The diagram being shown in accordance with one or more example embodiments of the present disclosure.

FIG. 7 illustrates a functional diagram of an example illustrated by the cross markers depicted peaks as detected in the compressed signal (vertical bars). The lines connecting the markers from an individual mass spectrum to another depict the detected ridges, in accordance with one or more example embodiments of the present disclosure.

FIG. 8 is a block diagram of an example of a mass spectrometer (MS) system disclosing such system elements in accordance with one or more example embodiments of the present disclosure,

FIG. 9 is a block diagram of an example of an analyzer hardware of an embodied mass spectrometer (MS) system as system elements, in accordance with one or more example embodiments of the present disclosure,

FIG. 10 is a block diagram of an example of a deconvolution algorithm in an embodied mass spectrometer (MS) system in accordance with one or more example embodiments of the present disclosure,

FIG. 11 is a block diagram of an example of a LASSO-algorithm variant of an embodied deconvolution algorithm in FIG. 10, in accordance with one or more example embodiments of the present disclosure,

FIG. 12 is an illustration of examples of dictionary atoms generated for specific targets, including all significant isotopes, in accordance with one or more example embodiments of the present disclosure,

FIG. 13 is an illustration of examples of diversified segmentation analysis being made in accordance with one or more example embodiments of the present disclosure, and

FIG. 14 illustrates a multi-core microprocessor configured to a segmentation originating analysis in accordance with one or more example embodiments of the present disclosure, and in option or in supplement a neural network of the cores.

### DETAILED DESCRIPTION

Example embodiments described herein provide certain systems, methods, and devices for peak location determination from a mass spectrometer signal in accordance of the disclosure of the embodiments.

The following description and the drawings sufficiently illustrate specific embodiments to enable those skilled in the art to practice them. Other embodiments may incorporate structural, logical, electrical, process, and other changes. Portions and features of some embodiments may be included in, or substituted for, those of other embodiments. Embodiments set forth in the claims encompass all available equivalents of those claims.

A method of processing a mass spectrometer signal associated to the ion masses in an ensemble of mass spectra determined by the mass spectrometer in accordance of this disclosure comprises:
segmentation of the spectra,
compression of the data points,
peak finding from the spectra
ridge detection, at the adjacent peaks represented by consecutive spectra,
peak data processing,
identification of peaks,
identifying the chemical information of the abundances,
classification of the findings,
in addition or optionally, in a suitable phase to maintain the mass spectrometer library and/or the calibration of the hardware of the mass spectrometer, in which machine learning and target detection/identification can be used.

According to an embodiment of the disclosure, a deconvolution algorithm as embodied obeys the signal processing method according to one or more embodiments of the disclosure.

Mass spectrum consists of a positive linear combination of peaks corresponding to specific ions present in the sample. The desired output from the signal-processing algorithm is an array of peaks containing their positions and intensities, from which chemical information can be extracted. Peak detection is still a major challenge in many MS-based analysis methods, particularly those where detection of peaks with low amplitudes is of importance.

Finally, in order to acquire chemical information from the peak data, the peaks need to be identified. That is, they need to be assigned an elemental composition from a predefined instrument library.

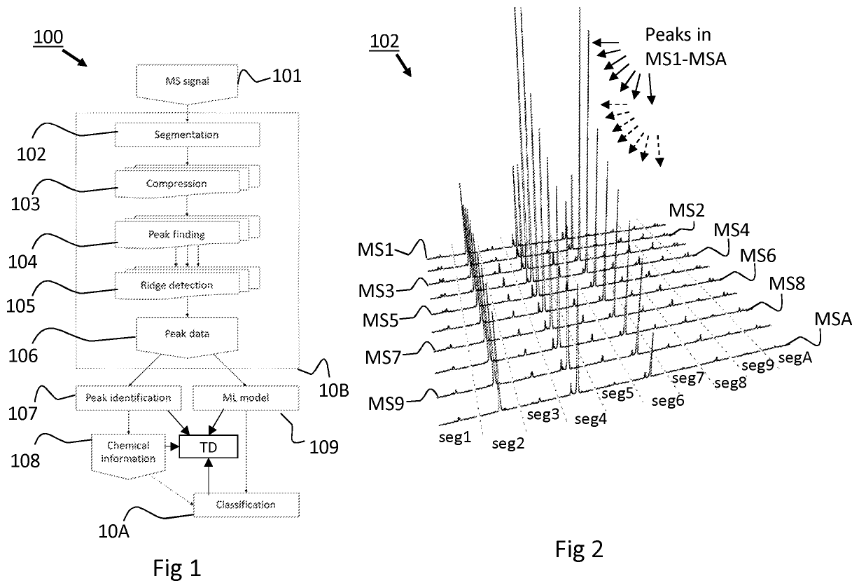

The presented algorithm addresses all fore mentioned challenges related to MS signal processing and peak identification. Overview of the algorithm operation is depicted in FIG. 1, where the signal processing part is marked out with a dashed rectangle. First, the signal (spectrum) is sliced into segments of a few hundred data points. The segments are independent of each other, and thus can be processed in parallel. Compression algorithm is applied to each segment, reducing the memory and computational cost in further processing. An important feature of the compression algorithm is its capability of enhancing the effective resolution for peak detection. The next step is to find peaks from the compressed signal. Finally, once all the spectra in an experiment have been processed, ridge detection is applied to the peak finder results and final peak array is constructed.

The results of the signal processing—the peak data—can be used as such for extracting chemical information about the sample. This requires a predefined instrument library, towards which the detected peaks can be compared and identified. The other option to utilize the peak data is through machine learning models. There the idea is not to explicitly identify certain chemical compounds, but rather use large datasets (many samples) to train a model that can solve the classification task.

In the following, all the said processing steps are discussed in a more detail in accordance of the disclosure. Processing steps can be implemented by means of software routine each, in suitable parts.

Segmentation according to an embodiment it is a first step of the signal processing, after having an ensemble of mass spectra obtained by the mass spectrometer, or when retrieved form a memory of the mass spectrometer device itself or a functionally connected data storage thereto for the purpose. The signal (mass spectrum) can be split to independent segments to enable parallel computing, and thus the processing speed to scale with the number of available CPU cores. This allows near real-time signal processing in applications where it is desired, since the cost of a multi-core computer is typically negligible compared to the high-resolution mass spectrometer itself. According to an embodiment, the CPU cores can be diversified over a network structure to connect many computers for operating as they were forming computing neurons to operate each for a segment of the same ensemble of mass spectra under processing.

Signal compression in the embodied method is based on sparse coding, where the purpose is to find a sparse representation of the signal in the form of a linear combination of dictionary atoms. The assumption is that mass spectra consist of a positive linear combination of peaks, and therefore the dictionary should consist of the expected peak shapes. The sparse coding problem is then solved by Lasso algorithm. According to an embodiment variant, by using coordinate descent.

Peak finding in the embodied method can be made by a simple local maxima finder according to an embodiment of the disclosure that is used to find peaks in the code, representative of the masses in the mass spectrometer results in the sample analysis. According to an embodiment of the disclosure, the peak finder is run for all segments in the sequence and a peak matrix is acquired as a result to be used in the further steps of the embodied method.

Ridge detection according to an embodiment of the disclosure, comprises also that physically meaningful peaks are expected to be present in many consecutive spectra (at an adjacent location on mass axis), while peaks that appear in just one or a few spectra are considered noise. In an embodiment according to the disclosure, ridge detection as such is applied to the peak matrix holding all the found peaks in a sequence, in order to filter out the noise. According to an embodiment of the disclosure, lines connecting the found local maxima in the code are determined and found for illustrative purposes shown to a user.

Peak identification by using a very important peak identification criterion according to an embodiment of the disclosure is obviously the mass-to-charge ratio of the detected peak. However, often more than one peak in the mass spectrum are associated with a certain chemical compound in the sample. This can be due to the isotopic patterns of elements in the compound, fragmentation of the parent molecule into multiple smaller ions, or the molecule being ionized by different ionization mechanisms simultaneously. The intensities of ion signals originating from the same parent molecule are expected to correlate in time, and the expected isotopic ratio is known. The ratio of fragments or ions formed by different mechanisms can be inferred from calibration.

Machine learning model can be used in creating an instrument library and adding new compounds to it is typically work intensive as such. Especially in the case where the ionization mechanism of the mass spectrometer system is such that multiple ions correspond to a single parent molecule. On the other hand, sometimes the samples to be analyzed are chemically so complex that it is not feasible to even try to explicitly identify all the compounds in the spectrum.

A way to utilize more of the information in the mass spectra (MS) is to use machine learning (ML) models according to an embodiment of the disclosure. However, it would be unpractical to train the models on raw MS signals, since they are noisy, and the dimensionality is unreasonably high. In general, the number of samples in a training data set for an ML model should be significantly greater than the number of features in each sample. The dimensionality of a high-resolution mass spectrum can be greater than 100,000, which means that the number of samples should be close to a million, typically not achievable.

The described signal processing and peak detection routine is essentially a method of feature extraction, since the peaks in the spectra are the features of interest. Training ML models on the peak data rather than raw signals reduces the number of features significantly, by a factor of >100. Thus, the minimum size of a training dataset to train reliable machine learning model would be in the order of thousands of samples, already a realistic number for practical applications.

Target detection can be used in a special cases where the goal is to detect specific target compounds from the mass spectrum, instead of more generic peak detection as such in accordance of the embodiments of the disclosure, it can be useful to define the segmentation borders and peak dictionary differently than described above as embodied for the generic peak detection.

The above descriptions are for purposes of illustration and are not meant to be limiting. Numerous other examples, configurations, processes, etc., may exist, some of which are described in greater detail below. Example embodiments will now be described with reference to the accompanying figures.

FIG. 1 is showing a flow-type diagram to illustrate a signal processing method 100 according to some example embodiments of the present disclosure. In an embodied signal processing method 100 it comprises:
segmentation of the spectra 102,
compression of the data points 103,
peak finding from the spectra 104
ridge detection 105, at the adjacent peaks represented by consecutive spectra,
peak data processing 106,
identification of peaks 107,
identifying the chemical information 108 for the substances and their abundances, and/or
classification of the findings 10A.

According to a further variant of the embodiment, the signal processing method 100 can comprise in addition or optionally, in a suitable phase to maintain the mass spectrometer library and/or the calibration of the hardware of the mass spectrometer, by a machine learning (ML) model.

According to a further variant of the embodiment the signal processing method 100 can comprise target detection can be also used for detecting is a certain substance (for example a hazardous or otherwise interesting substance) present.

In an embodiment, a mass spectrometer is used to analyze a sample to find out the composition and/or the abundances in the sample. The mass spectrometer so provides a mass spectrometer MS signal 101, in which atomic masses of the substances present in the sample each are shown in the spectrum, so that the abundance of the substance is proportional to the shown peak height at the corresponding substance mass and/or mass to charge ratio.

Segmentation 102 as shown in FIG. 1 and illustrated in FIG. 2, where an example of an ensemble of spectra with segmentation 102 as determined by a mass spectrometer are illustrated with the reference to FIG. 2. The mass spectra shown are MS1, MS2, MS3, MS4, MS5, MS6, MS7, MS8, MS9 and MSA. As the FIG. 2 illustrates, the spectra are a result from a repeated scan of the sample, and indicate peaks in each with a variation, as determined from the peak height differences of the adjacent peaks in the corresponding spectra.

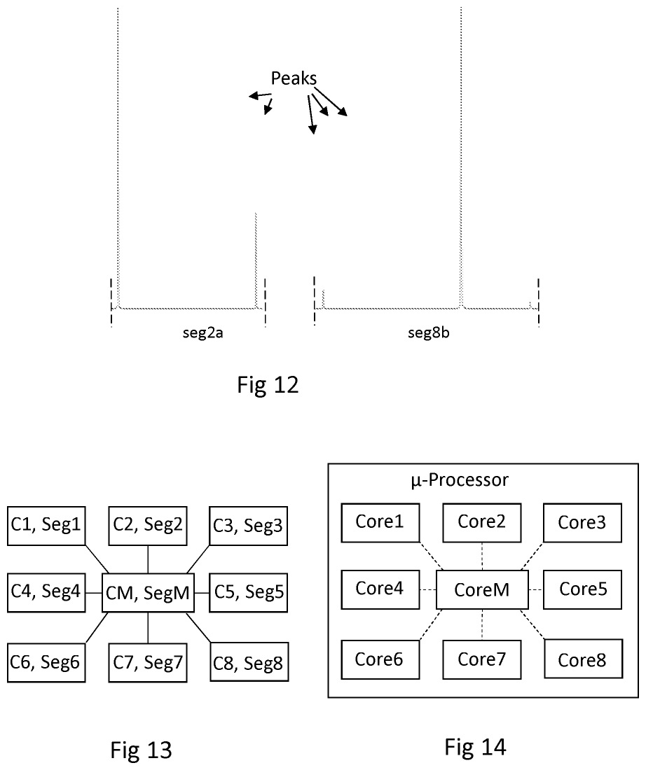

As illustrated in FIG. 1, in the algorithm following the method 10B, the spectra of FIG. 2 are divided to the indicated segments in the example of FIG. 2. The indicated segments are seg1, seg2, seg3, seg4, seg5, seg6, seg7, seg8, seg9, segA, and in FIG. 12 seg2a, and seg8b in relation the embodiment example shown therein. The segmentation is made in the method step 102 as indicated in FIG. 1.

Segmentation 102 can be made after having an ensemble of mass spectra obtained by the mass spectrometer MS, or when retrieved form a memory of the device itself or a functionally connected data storage for the purpose, the signal (mass spectrum) can be split to independent segments seg1-segA, which are illustrated in the FIG. 2 and FIG. 12 by dashed lines, to enable parallel computing, and thus the processing speed to scale with the number of available CPU cores. This allows near real-time signal processing in applications where it is desired, since the cost of a multi-core computer is typically negligible compared to the high-resolution mass spectrometer itself. According to an embodiment, the CPU cores can be diversified even over a network structure as illustrated in FIG. 13 embodiment, to connect many computers as neurons (C1, C2, C3, C4, C5, C8, CM with the respective segments seg1, seg2, seg3, seg4, seg5, seg6, seg7, seg8, segM), for operating as they were forming computing neurons to operate each for a segment of the same ensemble of mass spectra under processing.

The number of Mass spectra in FIG. 2 as shown therein represents an example of a solid number of spectra in an embodiment directed to solid batch embodiment, to process an ensemble of spectra having a predetermined number of spectra. A gliding batch can be also illustrated via the FIG. 2, if considered that for example spectrum MSA were assumed to be added to the ensemble of previously measured spectra MS1 to MS9 during operation of the mass spectrometer in an ongoing analysis. The segmentation as well as compression, peak finding as well as ridge detection can be updated in the gliding batch according to the progress of the measurement and the number of spectra being acquired. So, this embodiment can be used in measurements on-line, where there can be many spectras being acquired from same sample, in a certain way forming cumulative data.

According to an embodiment of the disclosure, each computer C1 . . . CM can have a number of cores, to be used in the segment analysis, so that a computer can analyze one or more segments, in accordance of the core number. The way of drawing illustrates also capabilities of the computers, as the CM illustrates a master computer that can handle all the rest of the spectrum segments and/or to draw together the results from the signal processing of the segments in accordance of one or more examples of embodiments of the present disclosure.

According to an embodiment of the disclosure, different strategies can be used to define the segment borders. One way to split the spectrum where there are no peaks as identified predetermined way, even though it is not critical. A simple way is to define the segments as (u−0.5, u+0.5), where u is an integer m/z, as depicted in the disclosure. This works well for relatively light (<500 Th) singly charged ions. For some applications, it may be desirable to define the borders differently to achieve optimal results in such embodiments.

This kind of a diversification as embodied may be applicable if there are for example mobile mass spectrometers, situated into a vehicle for example, to be used in detection of the same sample in suitable part, or, being used in identification of a certain particular substances in a certain segment, the presence and/or the abundance in the sample of the corresponding mass spectrometer. According to an embodiment example, the spectra can be acquired by an ensemble of corresponding mass spectrometers, and the analysis/identification has been diversified according to the divided segmentations.

According to the disclosure, different strategies can be used to define the segment borders. One way to split the spectrum where there are no peaks as identified predetermined way, even though it is not critical. A simple way is to define the segments as (u−0.5, u+0.5), where u is an integer m/z, as depicted in the disclosure. This works well for relatively light (<500 Th) singly charged ions. For some applications, it may be desirable to define the borders differently to achieve optimal results in such embodiments.

The diversification can be made by using a suitable network. According to an embodiment, a wireless network may include one or more computers as depicted in the example of FIG. 13, which may communicate in accordance with IEEE 802.11 communication standards, in such embodiment. The FIG. 13 computers may be mass spectrometers in mobile devices that are non-stationary (e.g., not having fixed locations) or may be mass spectrometers in stationary devices.

Compression 103 as shown in FIG. 1 as followed to the segmentation 102 being made as embodied. Although FIG. 1 shows the compression 103 to follow the segmentation 102 as such, in an embodiment of one or more embodiments in accordance of the disclosure, compression 103 and segmentation 102 may be somewhat parallel for some segments in respect to some other segments being headed to the compression while the segmentation were in processing. In other words, the cores in which the segmentation has been intended to be made may be used also in compression, and dependently on the segmentation related work load of a core in an ensemble of cores, they may be in different timing, the master computer or a core operating as such in a single computer system, is configured to wait until the next step can be performed.

In an embodiment, the optimization problem can be formulated as:

According to the disclosure, the signal is formed by a code and a dictionary parts:

signal˜=code*dictionary,

where the goal in accordance of the disclosure is to find the (non-negative) code. According to the embodied disclosure, approximation of the original signal can then be reconstructed by multiplying the dictionary by the code. Non-zero values in the code represent the likelihood of a peak representing an abundant substance in the mass spectrum, being present in embodiments of the disclosure.

The code representation of the signal in accordance of the disclosure has at least two key benefits. Firstly, it is sparse and as such has small memory footprint; sparse coding typically provides compression ratio greater than 10 without significant information loss. Secondly, it significantly enhances the effective resolution for peak detection, in accordance of the disclosure.

An important feature of the dictionary generation in accordance of the disclosure is, that utilizing a priori information about the instrument characteristics (namely the transmission and resolution functions of the MS, determined by calibration), becomes easy by the embodiments of the disclosure.

According to an embodiment of the disclosure, at first, the ion transmission function of the mass spectrometer (“peak shape”) is experimentally determined. Notably, it can be defined as an arbitrary 1D vector in accordance of an embodiment of the disclosure, in addition or instead of an analytic function (e.g. Gaussian, Lorentzian) as required by model-based peak detection methods as such.

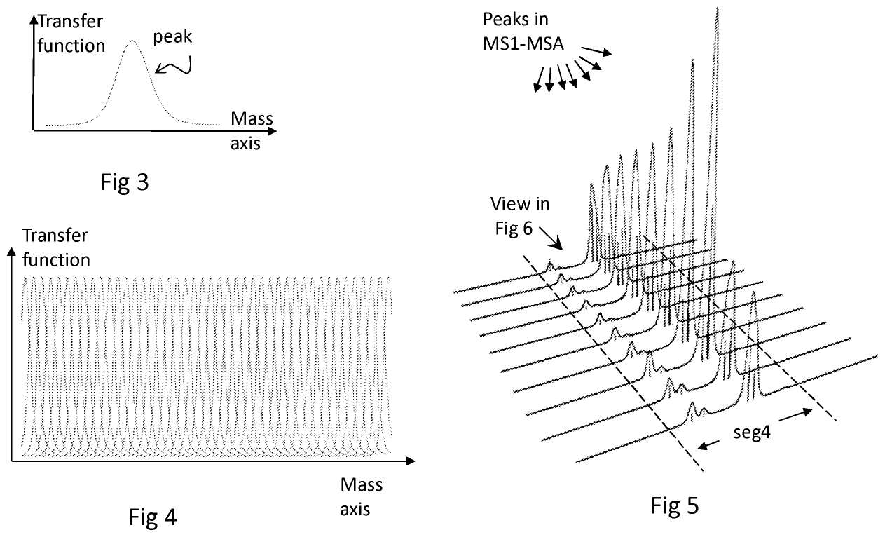

An example of a peak shape is shown in FIG. 3. The peak shape typically varies along the mass axis, as the transfer function value is represented as a function of mass, or mass/charge ratio. In traditional methods as such, only the variation in peak width is considered, as described by the resolution function of such a mass spectrometer. In the method described by the embodied disclosure here in accordance of the embodiments of the disclosure, a peak shape can be quite freely defined for each discrete point in the mass spectrum, so enabling compensation for non-idealities of the embodied mass spectrometer's hardware parts. Therefore, the calibration may limit the precision, not the applicability of a certain function to describe the actual peak shape. The peak shapes are used as dictionary atoms with the related mass when solving the sparse coding problem in accordance of the disclosure.

When constructing the dictionary of the dictionary atoms with their representative masses, in accordance of the disclosure, typically one atom per discrete signal sample is generated. Then, if a single mass spectrum consists of N samples, the dictionary is an N×N matrix, where each row corresponds to a single atom in accordance of the embodied disclosure. An arbitrary row i can be thought of as a 1D vector representing a peak whose maximum is at the index i. However, the resolution of the dictionary can be increased by adding atoms whose maximum is not at i or i+1 but i+1/k. In this case, in accordance of the disclosure, the size of the dictionary would become kN×N.

This is called supersampling by a factor of k in accordance of an example of embodiments of the disclosure. Supersampling is beneficial in accordance of the disclosure in such embodiment where the sampling rate of the mass spectrometer data acquisition card would otherwise limit the m/z (mass to charge) precision of peak detection, i.e. supersampling allows the peak position to fall in between two discrete samples as embodied in the disclosure

According to an embodiment of the disclosure, the dictionary matrix is parse (density in the order of 0.01%, according to an embodiment example) and despite its high dimensionality can be efficiently stored in a computer memory, or in suitable parts to computers in a diversified embodiment of the disclosure.

According to an embodiment of the disclosure, the compression is applied to each segment separately. A regularization parameter alpha needs to be selected such that desired output is achieved, that is, the balance between reconstruction error and sparsity as in accordance of the embodiment of the disclosure. For the same value of alpha to be suitable for each segment, they need to be normalized to unit norm before processing in an embodiment variant of the disclosure. According to an embodiment of the disclosure, the regularization parameter alpha is considered as an estimate being made dedicated means to calculate the parameter for each regularization occurrence.

According to an embodiment of the disclosure, the compression algorithm as such can enhance the resolution and the overlapping peaks can be separated accordingly.

FIG. 4 illustrates how an embodied dictionary is constructed of individual peak shapes representing the corresponding masses. In FIG. 4, only atoms of every 10th sample (mass) are shown for the sake of clarity of the drawing. Most of the values on each row are zeros, since they only hold the information of a single peak. Thus, the dictionary matrix is sparse (density in the order of 0.01%) and despite its high dimensionality can be efficiently stored in computer memory or into an ensemble of suitable computers in a diversified embodiment of signal processing.

Peak finding 104 can be made as based on the compression 103 results. An example of compression results for a sequence of ten segments of mass spectra is depicted in FIG. 5 at a segment seg4. The spectra are not necessarily the same as in the example of FIG. 2. There is a large high peak that is a superposition of two overlapping peaks. In addition, there are two smaller peaks. The vertical bars at the peak locations on the mass axis depict the compression results—the “code”. It can clearly be seen how the compression algorithm enhances the resolution, and the larger overlapping peaks become well separated. The compression ratio (nsignal>0/ncode>0) in this example is 32.

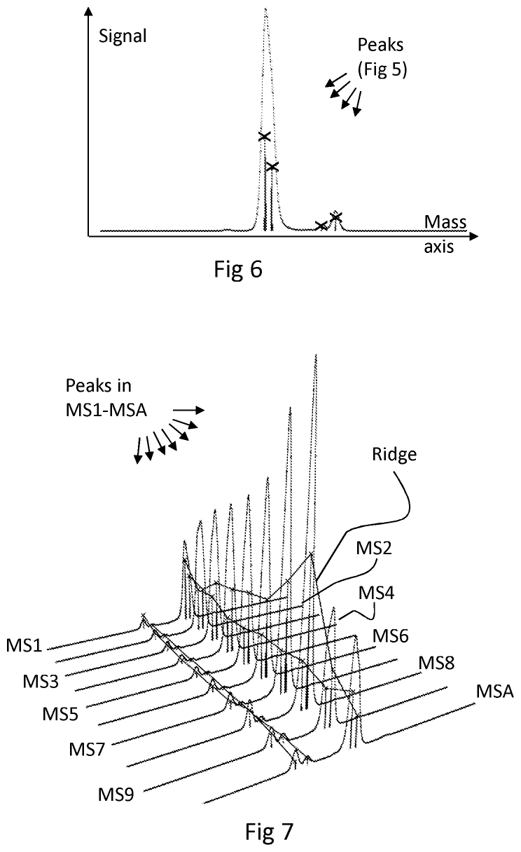

The wording shown as “View in FIG. 6” is illustrative to observe the same segment of FIG. 5 as it were seen in FIG. 6.

Example of a single signal segment (seg4, FIG. 5) and its associated code is depicted in FIG. 6, where four found peaks are shown by cross markers at the top of the representative vertical line. The peak finder is run for all the segments in the sequence, and a peak matrix is acquired as a result.

Ridge detection 105 is illustrated as shown in FIG. 7, local maxima of each spectrum are depicted by cross markers in the code representing vertical bar and the detected ridges by lines connecting the markers are drawn to illustrate the ridges (as for example the Ridge in FIG. 7).

According to an embodiment of the disclosure, physically meaningful peaks are expected to be present in many consecutive spectra (at an adjacent location on mass axis), while peaks that appear in just one or a few spectra are considered noise. According to an embodiment, the algorithm operating according to the method has a dedicated means to observe ridges, ridge detector. According to an embodiment, the ridge detector is configured also to find from locations on mass axis that a ridge would have a zero values or near zero values. In case of repetitive observation of zeros in consecutive mass-corresponding locations, the ridge detector is configured to consider the mass related peak as noise when there are more zeros values in the same ridge in a consecutive manner than a threshold parameter defined to control the ridge detector operation to detect noise.

In an embodiment according to the disclosure, ridge detection 105 as such is applied to the peak matrix holding all the found peaks in a sequence, in order to filter out the noise. According to an embodiment of the disclosure, lines connecting the found local maxima in the code are determined and found for illustrative purposes shown to a user.

According to an embodiment of the disclosure, there are three parameters controlling the ridge detection, distance threshold, gap threshold and minimum length. According to an embodiment the distance threshold defines the maximum distance (in m/z space) of two peaks in consecutive segments that are to be considered part of the same ridge. According to an embodiment, the gap threshold can be used to allow a gap in the ridgeline, i.e. one or more segments within the ridge where the peak was not found (within the distance threshold). According to an embodiment, the minimum length defines the minimum number of consecutive (within gap threshold) peaks required in the ridgeline to be considered a ridge. Each ridge is considered to represent a certain ion present in the sample, as embodied in accordance of the disclosure.

In Peak identification 106, therefore, according to an embodiment of the disclosure, three peak identification criteria are applied: 1) peak m/z; 2) peak intensity ratio; 3) peak intensity correlation.

For the first criterion, m/z of the detected peak is compared in an embodiment with the compounds in the instrument library in accordance of the embodiment of the disclosure, and if a match is found within a set tolerance, the criterion is met.

If all peaks corresponding to a certain compound in the library have been detected according to an embodiment, as based on the m/z criterion, their signal intensity ratio is compared in an embodiment with the expected ratio. According to an embodiment variant, the ratio must be within a set threshold to meet the criterion embodied.

Lastly, according to an embodiment of the disclosure, a Pearson correlation coefficient for the time series of the set of peaks is calculated according to an embodiment, and the coefficients between all the peaks must match a predefined threshold as in accordance of an embodiment variant of the disclosure.

If all the criteria are met, as embodied, the peak set is considered identified and the compound they correspond to be detected 108. This information can be used according to an embodiment of the disclosure as such for classification (i.e. to determine is a certain chemical present in the sample or not). The results can be classified 10A according to a predetermined criterion in accordance of the interest to the substances wanted to be detected.

Machine learning model 109 has been indicated in FIG. 1, to be considered as a way to utilize more of the information in the mass spectra (MS) in the signal processing is to use machine learning (ML) models according to an embodiment of the disclosure. However, it would be unpractical to train the models on raw MS signals, since they are noisy, and the dimensionality is unreasonably high. In general, the number of samples in a training data set for an ML model should be significantly greater than the number of features in each sample. The dimensionality of a high-resolution mass spectrum can be greater than 100,000, which means that the number of samples should be close to a million, typically not achievable.

The described signal processing and peak detection routine is in a certain way a method of feature extraction, since the peaks in the spectra are the features of interest. Training ML models on the peak data rather than raw signals reduces the number of features significantly, by a factor of >100. Thus, the minimum size of a training dataset to train reliable machine learning model would be in the order of thousands of samples, already a realistic number for practical applications.

According to an embodiment of the disclosure, the machine learning can be used in the mass spectrum segments, as in a diversified manner in diversified segments using cores of the microprocessors.

Target detection TD as shown as an example in FIG. 1, where according to an embodiment of the disclosure, for each target, a peak kernel is generated by considering the m/z and isotopic pattern of the target (a transfer function with shape corresponding the isotope of a dictionary atom), and the dictionary is constructed of these kernels. In FIG. 12, two examples of such peak kernels are shown. When using an embodied dictionary built in this way as embodied, also the segmentation needs to be defined accordingly. That is, the segment borders are defined for each target such that all the significant isotopes are included into the segment under the interest, instead of just one unit mass.

The benefit of defining the dictionary in this way according to an embodiment variant of the disclosure is that the peak identification step becomes unnecessary in certain sense. Ridge detection is applied to the peak kernels similarly as with individual peaks according to an embodiment, and when/if a ridge is detected, it already means that the peak identification criteria (m/z, isotopic ratio, isotope correlation) have been met and the target is detected according to the embodiment of the disclosure. According to the embodiment variants in accordance of the disclosure, chemical information 108 can be used in the target detection in suitable part, but also in addition or option, also machine learning with peak identification in combination or alone in suitable part for the sample analysis constituting the mass spectrum data to processed. Classification routines can be used in suitable part to classify targets according to the recognition of the ridges.

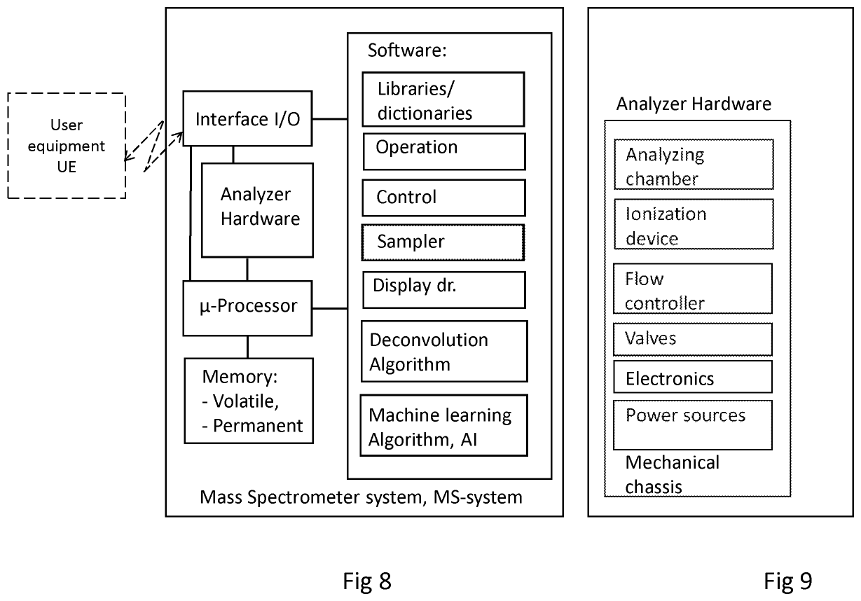

FIG. 8 illustrates in accordance of the disclosure a mass spectrometer system, an MS-system that includes therein as system elements as the items depicted, such as the analyzer hardware, a microprocessor (μ-processor) and a memory, which can have volatile and permanent memories in use of the microprocessor, especially to be used in control of the hard ware of the MS-system in the acquiring spectra. The volatile memory has been addressed to the algorithms such as the signal processing algorithm obeying the embodied method, as well as the parameters in use of the hardware control. The permanent memory is also in use of the microprocessor for saving the spectra as well as the control parameters and settings as well as user profiles. In the memory, there can be also the software involved in suitable part.

As non-limiting example the mass spectrometer system software in the software block (Software) can comprise dictionaries for the signal processing method, operation related software to be used in the control according to the Control-indicated control parameters. The software can comprise also operational parameters for the hardware control in the sampling, drivers for example for display, operation voltages their regulation schemes but also deconvolution algorithms, to operate in accordance of the embodiments of the disclosure about the disclosed signal processing method of a mass spectrometer. In addition, software related subroutines of the algorithm to obey the embodied method 100 according to the method steps (102-10A) in FIG. 1 can be stored in the memory. According to an embodiment the software block is a system element, as well as the analyzer hardware as further embodied in the FIG. 9.

The storing in the part of the permanent memory is intended for a longer storage as when the MS-system's microprocessor is in an off state. Also for survival of power loss, to save the state of the MS-system and parameters at the very moment of operation at the power loss. The volatile memory is considered as a working memory when at least the microprocessor is operation in the MS-system. Machine learning algorithms as well as artificial intelligence AI, related algorithms can be stored in the memory, the memory part of which being determined is the algorithm in the use of the microprocessor currently, or is it in an off-state.

The interface I/O is also illustrating access of the microprocessor to the software in the MS-system. According to an embodiment, the access to the software and the related operations are performed in the control of the microprocessor to maintain the operations of the MS-system, or at least a suitable part of it, according to the user's interest. The microprocessor can be a multicore microprocessor, as illustrated for the embodiments in accordance of the instant disclosure with reference to FIG. 14.

In addition, according to an embodiment variant of the disclosure, the interface I/O may have a transceiver to be used in the outsourcing the mass spectrometer controls so that a portable device, user equipment UE, can communicate with the mass spectrometer system, in order to control the operation and/or retrieve the spectrum-related data. The transceiver in the UE (FIG. 8) can be compatible with Wi-Fi, Bluetooth, BT, IP, LAN, WAN or another wireless protocol. Such a mass spectrometer can be associated to an IoT-network, so to facilitate the use of the internet of things, and in suitable part, the intelligence of the IoT network comprising microprocessors as neural network nodes. Corresponding manner, in such an embodiment also the interface I/O is provided with compatible transceiver to facilitate the communication accordingly.

As used herein, the term “Internet of Things (IoT) device” is normally used to refer to any object (e.g., an appliance, a sensor, etc.) that has an addressable interface (e.g., an Internet protocol (IP) address, a Bluetooth identifier (ID), a near-field communication (NFC) ID, etc.) and can transmit information to one or more other devices over a wired or wireless connection, provided with a suitable transceiver. An IoT device may have an active communication interface (I/O, FIG. 8), such as a modem, a transceiver, a transmitter-receiver, or the like. An IoT device can have a particular set of attributes (e.g., a device state or status, such as whether the IoT device is on or off, open or closed, idle or active, available for task execution, such as a computation in accordance of the disclosure of a diversified segmentation, or busy, and so on, that can be embedded in and/or controlled/monitored by a central processing unit (CPU), microprocessor, ASIC, or the like, and configured for connection to an IoT network such as a local ad-hoc network or the Internet. The MS-system computer can be used in diversifying the suitable tasks, i.e. segments related signal processing to other devices in connection.

Accordingly, the IoT network may be comprised of a combination of “legacy” Internet-accessible devices (e.g., laptop or desktop computers, cell phones, etc.) in addition to devices that do not typically have Internet-connectivity.

The user device(s) may also include mesh stations in, for example, a mesh network, in accordance with one or more IEEE 802.11 standards.

The user interface UE (FIG. 8) may be a personal computer (PC), a tablet PC, a set-top box (STB), a personal digital assistant (PDA), a mobile telephone, a wearable computer device, a web appliance, a network router, a switch or bridge, or any machine capable of executing instructions (sequential or otherwise) that specify actions to be taken by that machine, such as a base station. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), or other computer cluster configurations.

The storage device as a memory in FIG. 8 may include a machine readable medium on which is stored one or more sets of data structures or instructions (e.g., software) embodying or utilized by any one or more of the techniques or functions described herein for the mass spectrometer operation and/or signal processing of mass spectrometer spectra in accordance of the method exemplified in FIG. 1. The instructions may also reside, completely or at least partially, within the permanent memory, within the volatile memory, or within the hardware processor's dedicated working memory during execution thereof by the microprocessor provided with one or more cores. In an example, one or any combination of the hardware processor, the permanent memory, the volatile memory, or an external storage device may constitute machine-readable media. While the machine-readable medium is illustrated as a single medium, the term “machine-readable medium” may include a single medium or multiple media (e.g., a centralized or distributed database, and/or associated caches and servers) configured to store the one or more instructions.

Various embodiments may be implemented fully or partially in software and/or firmware. This software and/or firmware may take the form of instructions contained in or on a non-transitory computer-readable storage medium. Those instructions may then be read and executed by one or more processors to enable performance of the operations described herein. The instructions may be in any suitable form, such as but not limited to source code, compiled code, interpreted code, executable code, static code, dynamic code, and the like. Such a computer-readable medium may include any tangible non-transitory medium for storing information in a form readable by one or more computers, such as but not limited to read only memory (ROM); random access memory (RAM); magnetic disk storage media; optical storage media; a flash memory, etc.

The term “machine-readable medium” may include any medium that is capable of storing, encoding, or carrying instructions for execution by the machine and that cause the machine to perform any one or more of the techniques of the present disclosure, or that is capable of storing, encoding, or carrying data structures used by or associated with such instructions concerning the signal processing of the mass spectrometer of the MS-system. Non-limiting machine-readable medium examples may include solid-state memories and optical and magnetic media. In an example, a massed machine-readable medium includes a machine-readable medium with a plurality of particles having resting mass. Specific examples of massed machine-readable media may include non-volatile memory, such as semiconductor memory devices (e.g., electrically programmable read-only memory (EPROM), or electrically erasable programmable read-only memory (EEPROM)) and flash memory devices; magnetic disks, such as internal hard disks and removable disks; magneto-optical disks; and CD-ROM and DVD-ROM disks.

Example communications networks may include a local area network (LAN), a wide area network (WAN), a packet data network (e.g., the Internet), mobile telephone networks (e.g., cellular networks), plain old telephone (POTS) networks, wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, and peer-to-peer (P2P) networks, among others.

In FIG. 9, an analyzer hardware of a mass spectrometer system of FIG. 8 has been illustrated. In accordance of the instant disclosure as embodied, the analyzer hardware can have a mechanical chassis into which there can belong at least one of the following devices: an analyzing chamber, ionization device, a flow controller, valves, other electronic parts (i.e. power source to the system as such), Power sources, such as the high voltage power sources and/or magnetic field producing power sources, to mention few examples of such devices that can be comprised in the mass spectrometer system MS-system. According to an embodiment, at least one of these devices is operable in control of the microprocessor shown in FIG. 8 and/or in a core of such as shown in FIG. 14.

For example, some MS-system elements in FIG. 9 may include one or more microprocessors, DSPs, field-programmable gate arrays (FPGAs), application specific integrated circuits (ASICs), radio-frequency integrated circuits (RFICs) and combinations of various hardware and logic circuitry for performing at least the functions of the system part in the MS-system architecture described herein.

Certain embodiments of the mass spectrometer of a mass spectrometer-system may be implemented in one or a combination of hardware, firmware, and software. Other embodiments may also be implemented as instructions stored on a computer-readable storage device, which may be read and executed by at least one processor to perform the operations described herein. A computer-readable storage device (i.e. Memory, FIG. 8) may include any non-transitory memory mechanism for storing information in a form readable by a machine (e.g., a computer). For example, a computer-readable storage device may include read-only memory (ROM), random-access memory (RAM), magnetic disk storage media, optical storage media, flash-memory devices, and other storage devices and media.

Examples, as described herein, may include or may operate on logic or a number of components, modules, or mechanisms. Modules are tangible entities (e.g., hardware) capable of performing specified operations when operating. A module includes hardware. In an example, the hardware may be specifically configured to carry out a specific operation (e.g., hardwired). In another example, the hardware may include configurable execution units (e.g., transistors, circuits, etc.) and a computer readable medium containing instructions where the instructions configure the execution units to carry out a specific operation when in operation. The configuring may occur under the direction of the executions units or a loading mechanism. Accordingly, the execution units are communicatively coupled to the computer-readable medium when the device is operating. In this example, the execution units may be a member of more than one module. For example, under operation, the execution units may be configured by a first set of instructions to implement a first module at one point in time and reconfigured by a second set of instructions to implement a second module at a second point in time.

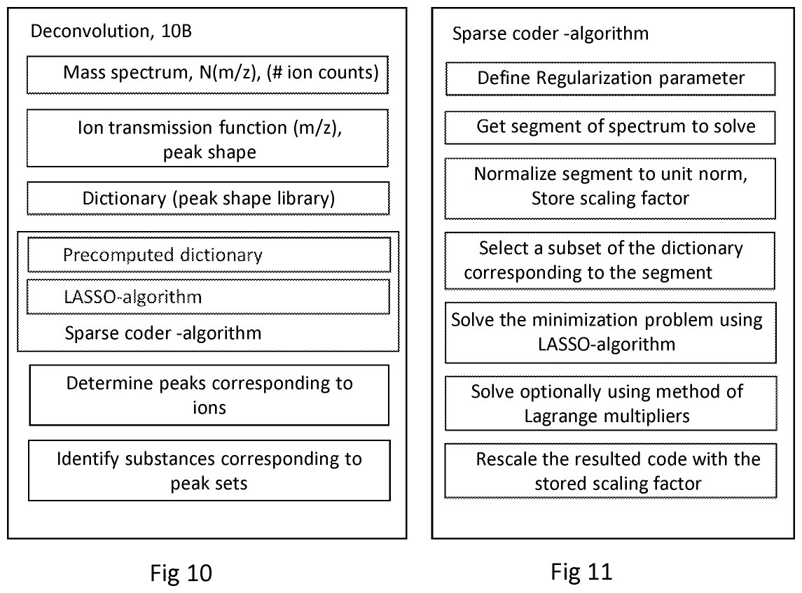

In FIG. 10 a deconvolution algorithm of the MS-system according to one or more embodiments of the instant disclosure, which algorithm has been considered as implementing the method 100 of FIG. 1. For the operation of such a deconvolution algorithm, an ensemble of mass spectra comprising at least one mass spectrum is acquired by the mass spectrometer, or retrieved from the memory connected to microprocessor of the MS-system.

In FIG. 10 example of embodiment variant of deconvolution (10B), the method comprises determination of Mass spectrum as number of ion counts for a certain ion mass, using in peak shape determination ion transmission function, with help of Dictionary having also a peak shape library as a part of a precomputed dictionary, The sparse code algorithm with LASSO and the precomputed dictionary are used, The peaks are determined corresponding to ions present in the sample, and the substances corresponding the peak sets are identified.

The algorithm may use the channels for the masses according to the dictionary to form the matrix as explained in the FIG. 1. Ion masses for various nuclides can be obtained from a library in the MS-systems memory, as well as the corresponding mobilities corresponding the nuclides with their masses. The algorithm forms the spectrum as a combination of the library nuclides as explained with reference to FIG. 1. According to an embodiment variant, the algorithm can also have a library for peak shape for the nuclides. According to an embodiment, the already observed peaks associated to a certain nuclide as found with the mass spectrometer hardware composition and parameters as a device set-up can be characterized by symmetry, skewness, peak width and/or other parameters to help identification of a certain peak from a ridge.

Although in FIG. 10 LASSO-algorithm has been indicated with reference only to sparse code solver and precomputed dictionary, the deconvolution algorithm in accordance of the method can have also other routines with LASSO algorithms.

Some features of a proprietary LASSO-algorithm variant, to be used in the method 100 in suitable part, are illustrated in FIG. 11. In such a variant embodiment, the number of spectrum and the nuclides therein can be defined as based on the library. The covariate matrix can be formed, and the vectors calculated to represent the spectrum and/or the ridges, in accordance of the covariate matrix calculation.

In an embodiment variant according to the FIG. 11, Sparse code algorithm comprises: Defining regularization parameter, getting segment of spectrum to solve, normalize segment to unit norm, and storing scaling factor(s), selecting a subset of the dictionary corresponding to the segment, solving the minimization problem using LASSO-algorithm, solving optionally using method of Lagrange multipliers, and Resolving the resulted code with the stored scaling factor. As an option, also a Lagranian solver can be used in suitable part in the minimization problem solving, for example, if LASSO would be suspected to be inappropriate for a certain data set of spectra. In the algorithm, peaks corresponding ion masses and then identify the substances.

FIG. 12 illustrates target detection from the indicated segments seg2a and seg8b, which are segments as in the illustration of FIG. 2 the respective segments seg2 and seg8, but are not necessarily the same, which being indicated by the respective letters a and b in the FIG. 12.

FIG. 13 illustrates diversification of the segments of FIG. 2 into different computers so that segment seg1 is handled in computer C1, segment seg2 is handled in computer C2, segment seg3 is handled in computer C3, segment seg4 is handled in computer C4, segment seg5 is handled in computer C5, segment seg6 is handled in computer C6, segment seg7 is handled in computer C7, segment seg8 is handled in computer C8. Similar way, in the example of FIG. 2 with 10 segments, the computer CM would be used in the operations to handle at least one of the segments seg9 and segA, if not resources were sufficient to both. Computer CM so illustrates that a one computer or its ensemble of cores can handle several segments. The above-cited computers C1 to CM illustrate also an example of different cores of one microprocessor as in FIG. 14 example also, but also possibility to divide the processing of different segments into diversified computer network, which according to an embodiment can form a neural network.

FIG. 14 illustrated a multicore microprocessor architecture example of a microprocessor in the MS-system embodiment to be used in one or more embodiments of the disclosure.

### About Generic Terms

The word “exemplary” is used herein to mean “serving as an example, instance, or illustration.” Any embodiment described herein as “exemplary” is not necessarily to be construed as preferred or advantageous over other embodiments. The terms “computing device,” “user device,” “communication station,” “station,” “handheld device,” “mobile device,” “wireless device” and “user equipment” (UE) as used herein refers to a wireless communication device such as a cellular telephone, a smartphone, a tablet, a netbook, a wireless terminal, a laptop computer, a femtocell, a high data rate (HDR) subscriber station, an access point, a printer, a point of sale device, an access terminal, or other personal communication system (PCS) device. The device may be either mobile or stationary.

As used within this document, the term “communicate” is intended to include transmitting, or receiving, or both transmitting and receiving. This may be particularly useful in embodiments when describing the organization of data that is being transmitted by one device and received by another, but only the functionality of one of those devices is required to describe operation of embodiment in question. Similarly, the bidirectional exchange of data between two devices (both devices transmit and receive during the exchange) may be described as “communicating,” when only the functionality of one of those devices is being embodied. The term “communicating” as used herein with respect to a wireless communication signal includes transmitting the wireless communication signal and/or receiving the wireless communication signal. For example, a wireless communication unit, which is capable of communicating a wireless communication signal, may include a wireless transmitter to transmit the wireless communication signal to at least one other wireless communication unit, and/or a wireless communication receiver to receive the wireless communication signal from at least one other wireless communication unit.

As used herein, unless otherwise specified, the use of the ordinal adjectives “first,” “second,” “third,” etc., to describe a common object, merely indicates that different instances of like objects are being referred to and are not intended to imply that the objects so described must be in a given sequence, either temporally, spatially, in ranking, or in any other manner.

Some embodiments with reference to user equipment may be used in conjunction with various devices and systems, for example, a personal computer (PC), a desktop computer, a mobile computer, a laptop computer, a notebook computer, a tablet computer, a server computer, a handheld computer, a handheld device, a personal digital assistant (PDA) device, a handheld PDA device, an on-board device, an off-board device, a hybrid device, a vehicular device, a non-vehicular device, a mobile or portable device, a consumer device, a non-mobile or non-portable device, a wireless communication station, a wireless communication device, a wireless access point (AP), a wired or wireless router, a wired or wireless modem, a video device, an audio device, an audio-video (A/V) device, a wired or wireless network, a wireless area network, a wireless video area network (WVAN), a local area network (LAN), a wireless LAN (WLAN), a personal area network (PAN), a wireless PAN (WPAN), and the like.

Some embodiments may be used in conjunction with one way and/or two-way radio communication systems, cellular radio-telephone communication systems, a mobile phone, a cellular telephone, a wireless telephone, a personal communication system (PCS) device, a PDA device which incorporates a wireless communication device.

Some embodiments may be used in conjunction with one or more types of wireless communication signals and/or systems following one or more wireless communication protocols, in the communication between the mass spectrometer system and user equipment, for example, radio frequency (RF), infrared (IR), frequency-division multiplexing (FDM), orthogonal FDM (OFDM), time-division multiplexing (TDM), time-division multiple access (TDMA), extended TDMA (E-TDMA), general packet radio service (GPRS), extended GPRS, code-division multiple access (CDMA), wideband CDMA (WCDMA), CDMA 2000, single-carrier CDMA, multi-carrier CDMA, multi-carrier modulation (MDM), discrete multi-tone (DMT), Bluetooth®, global positioning system (GPS), Wi-Fi, Wi-Max, ZigBee, ultra-wideband (UWB), global system for mobile communications (GSM), 2G, 2.5G, 3G, 3.5G, 4G, fifth generation (5G) mobile networks, 3GPP, long term evolution (LTE), LTE advanced, enhanced data rates for GSM Evolution (EDGE), or the like. Other embodiments may be used in various other devices, systems, and/or networks.

Certain aspects of the disclosure are described above with reference to block and flow diagrams of the method, and MS-system apparatuses, and/or computer program products according to various implementations. It will be understood that one or more blocks of the block diagrams and flow diagrams, and combinations of blocks in the block diagrams and the flow diagrams, respectively, may be implemented by computer-executable program instructions. Likewise, some blocks of the block diagrams and flow diagrams may not necessarily need to be performed in the order presented, or may not necessarily need to be performed at all, according to some implementations.

These computer-executable program instructions may be loaded onto a special-purpose computer (for example FIG. 13) or other particular machine, a processor (for example FIG. 14), or other programmable data processing apparatus to produce a particular machine with dedicated operations of the system embodied, such that the instructions that execute on the computer, processor, or other programmable data processing apparatus create means for implementing one or more functions specified in the flow diagram block or blocks. These computer program instructions may also be stored in a computer-readable storage media referred as memory (for example as described in FIG. 8) that may direct a computer or other programmable data processing apparatus to function in a particular manner, such that the instructions stored in the computer-readable storage media produce an article of manufacture including instruction means that implement one or more functions specified in the flow diagram block or blocks.

As an example, certain implementations may provide for a computer program product, comprising a computer-readable storage medium having a computer-readable program code or program instructions implemented therein, said computer-readable program code adapted execution to implement one or more functions specified in the flow diagram block or blocks of FIG. 1.

The computer program instructions may also be loaded onto a microprocessor or other programmable data processing apparatus to cause a series of operational elements or steps to be performed on the computer or other programmable apparatus to produce a computer-implemented process such that the instructions that execute on the computer or other programmable apparatus provide elements or steps for implementing the functions specified in the flow diagram block or blocks of a signal processing method of exemplified in FIG. 1.

Accordingly, blocks of the block diagrams and flow diagrams support combinations of means for performing the specified functions, combinations of elements or steps for performing the specified functions and program instruction means for performing the specified functions. It will also be understood that each block of the block diagrams and flow diagrams, and combinations of blocks in the block diagrams and flow diagrams, may be implemented by special-purpose, hardware-based computer in the mass spectrometer systems that perform the specified functions, elements or steps, or combinations of special-purpose hardware and computer instructions in accordance of the instant disclosure.

Conditional language, such as, among others, “can,” “could,” “might,” or “may,” unless specifically stated otherwise, or otherwise understood within the context as used, is generally intended to convey that certain implementations could include, while other implementations do not include, certain features, elements, and/or operations. Thus, such conditional language is not generally intended to imply that features, elements, and/or operations are in any way required for one or more implementations or that one or more implementations necessarily include logic for deciding, with or without user input or prompting, whether these features, elements, and/or operations are included or are to be performed in any particular implementation.

Many modifications and other implementations of the disclosure set forth herein will be apparent having the benefit of the teachings presented in the foregoing descriptions and the associated drawings. Therefore, it is to be understood that the disclosure is not to be limited to the specific implementations disclosed and that modifications and other implementations are intended to be included within the scope of the appended claims. Although specific terms are employed herein, they are used in a generic and descriptive sense only and not for purposes of limitation.

### Example 1

According to an embodiment of the disclosure, in an example of a use, the mass spectrometer of the mass spectrometer system can be used in breath analysis techniques, with the improved algorithm's capability to identify substances in relation to a model in a cancer detector from human breath samples, for example. The model is a binary classifier trained with labelled samples from both healthy people and cancer patients that can be identified better with the mass spectrometer using embodiments according to the disclosure. After training the embodied machine-learning embodiment, the machine-learning model will predict the class of a previously unseen sample. The model learns the significant features from the peak data, based on the training dataset, without the need for chemical identification of the peaks as such.

### Example 2

According to an embodiment of the disclosure, in an example of a use case, the mass spectrometer can be used in analyzing samples from for example airport security control, railway and/or boat as well as in customs at the boarders, in passenger control and entry as well as cargo handling, in detection of illicit substances that may be harmful in many ways. In addition, mobile laboratories can be equipped with a mass spectrometer that can use one or more of the embodiments of the disclosure.

### Example 3

According to an embodiment of the disclosure, in an example of a use, the mass spectrometer can be used in analyzing samples from for example an industrial process for traces of wanted and/or unwanted substances. For example clean room operations may be beneficial of the accurate substance identification according to one or more embodiments of the disclosure.

1. A signal processing method of mass spectrometer signal, indicative of mass spectrum of atoms abundant in the mass spectrometer analyzed sample, the signal processing method comprises:
segmentation of the spectra,
compression of the data points,
peak finding from the spectra
ridge detection, at the adjacent peaks represented by consecutive spectra,
peak data processing, and
identification of peaks.

2. The signal processing method of claim 1, wherein the method additionally comprises identifying the chemical information for the substances and the substances' abundances, and/or classification of the findings.

3. The signal processing method of claim 1, wherein the method comprises supersampling.

4. The signal processing method of claim 1, wherein the method comprises determining a supersampling factor in accordance of the dictionary matrix sample size.

5. The signal processing method according to claim 1, wherein the method comprises determining a Pearson correlation coefficient for the time series of the set of peaks.

6. The signal processing method of claim 5, wherein the coefficients between all the peaks are applied as a constraint to match a predefined threshold.

7. The signal processing method of claim 1, wherein the method comprises a machine-learning algorithm configured to operate as a feature extraction means in the peak detection and/or target detection.

8. The signal processing method according to claim 1, wherein the measured mass spectrometer spectra are processed by the signal processing method as a solid batch of spectra with a predetermined size to form a solid ensemble of spectra.

9. The signal processing method according to claim 1, wherein the measured mass spectrometer spectra are processed by the signal processing method as a gliding batch of spectra to an ensemble of before measured spectra to add a new spectrum so to increase the predetermined number of the ensemble of spectra.

10. The signal processing method of claim 8, wherein the method comprises updating at least one of the following: segment number, compression of the segments, the matrix size, the ridge detection and peak number.

11. A mass spectrometer configured to signal processing according to anyone of the signal processing method claim 1.

12. A non-transitory computer-readable medium storing computer-executable instructions which when executed by one or more processors result in performing operations comprising a signal processing method according to claim 1.

13. A mass spectrometer system comprising a software to provide a dedicated functionality to the mass spectrometer according to claim 11, in said mass spectrometer system, that comprises further as system elements analyzer hardware to acquire mass spectra, microprocessor for controlling the special functionality of the mass spectrometer with the analyzer hardware, memory and an I/O interface, in communication to other system elements, for mediating the control signals of the microprocessor according to the software block.

14. A mass spectrometer comprising a software to provide a dedicated functionality to the mass spectrometer, wherein the software to provide said dedicated functionality comprises computer-executable instructions stored on a non-transitory computer-readable medium which when executed by one or more processors result in performing the signal processing method according to claim 1.

15. The mass spectrometer system of claim 13, wherein the mass spectrometer system comprises a wireless information network access point for external control to control, a mass spectrometer of mass spectrometer system, by a user equipment.

16. The signal processing method of claim 2, wherein the method comprises supersampling.

17. The signal processing method of claim 2, wherein the method comprises determining a supersampling factor in accordance of the dictionary matrix sample size.

18. The signal processing method of claim 3, wherein the method comprises determining a supersampling factor in accordance of the dictionary matrix sample size.

19. The signal processing method according to claim 2, wherein the method comprises determining a Pearson correlation coefficient for the time series of the set of peaks.

20. The signal processing method according to claim 3, wherein the method comprises determining a Pearson correlation coefficient for the time series of the set of peaks.
